SEQUENCE LISTING (48 sequences)

SEQ ID NO: 1 - protein, 29 aa, Bacillus licheniformis
MNIKNIAKKASALTVAAALLAGGAPQTFA

SEQ ID NO: 2 - protein, 29 aa, synthetic construct
MNIKNIAKKASALTVAAALLAGGAPQASA

SEQ ID NO: 3 - protein, 29 aa, Bacillus licheniformis
MKQQKRLYARLLTLLFALIFLLPHSAAAA

SEQ ID NO: 4 - protein, 29 aa, synthetic construct
MKQQKRLYARLLTLLFALIFLLPHSAASA

SEQ ID NO: 5 - protein, 27 aa, Bacillus licheniformis
VKMLKKAVLIAAVFLLAAFAGSTEAFA

SEQ ID NO: 6 - protein, 27 aa, synthetic construct
MKMLKKAVLIAAVFLLAAFAGSTEASA

SEQ ID NO: 7 - DNA, 12437 bp, synthetic construct
  source 1..12437: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaatacattacattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacgcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 8 - protein, 483 aa, Bacillus licheniformis
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRYQETSGEYNIQAWTGFNFPGRGTTYSNWKWQWFHFDGTDWDQSRSLSRIFKFHGKAWDWPVSSENGNYDYLMYADYDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGAYDMRNILNNTLVASNPTKAVTLVENHDTQPGQSLESTVQPWFKPLAYAFILTRSGGYPAVFYGDMYGTKGTTTYEIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 9 - DNA, 9214 bp, synthetic construct
  source 1..9214: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaatacattacattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 10 - DNA, 3017 bp, Bacillus licheniformis
  source 1..3017: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccg

SEQ ID NO: 11 - DNA, 1320 bp, Bacillus licheniformis
  source 1..1320: mol_type = other DNA
ttgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataa

SEQ ID NO: 12 - DNA, 91 bp, synthetic construct
  source 1..91: mol_type = other DNA
gctgataaacagctgacatcaactaaaagtttcattaaatactttgaaaaaagttgttgacttaaaagaagctaaatgttatagtaattgt

SEQ ID NO: 13 - DNA, 58 bp, Bacillus subtilis
  source 1..58: mol_type = other DNA
acagaatagtcttttaagtaagtctactctgaatttttttaaaaggagagggtaaaga

SEQ ID NO: 14 - DNA, 34 bp, Bacillus licheniformis
  source 1..34: mol_type = other DNA
cggatttcctgaaggaaatccgtttttttatttt

SEQ ID NO: 15 - DNA, 3019 bp, Bacillus licheniformis
  source 1..3019: mol_type = other DNA
caagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 16 - DNA, 53 bp, synthetic construct
  source 1..53: mol_type = other DNA
catagtaccgagaaactagaggatccatcggacagctcttgcttgatatcttc

SEQ ID NO: 17 - DNA, 48 bp, synthetic construct
  source 1..48: mol_type = other DNA
gtcgtaatgatttttataatgacgcgtatcgcgttataccagtcatta

SEQ ID NO: 18 - DNA, 12526 bp, synthetic construct
  source 1..12526: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaatacattacattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtccgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 19 - DNA, 9515 bp, synthetic construct
  source 1..9515: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtcaacttttttgtcttcaccttcaatttctacctgcttaatatctttagcagcttctttataagaactttgaatatcgatcatcttgtccagtgttaagtttgttttgatgtttttttctaatgctgccaaaatctcttgatagtttgttaatgattttaattgtgcaccttcatgaataatttcgttgataatctcacgctgacgctcttgac

SEQ ID NO: 20 - DNA, 2991 bp, Bacillus licheniformis
  source 1..2991: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttac

SEQ ID NO: 21 - DNA, 1578 bp, Bacillus licheniformis
  source 1..1578: mol_type = other DNA
atgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaa

SEQ ID NO: 22 - DNA, 95 bp, synthetic construct
  source 1..95: mol_type = other DNA
gtcgctgataaacagctgacatcaatatcctattttttcaaaaaatattttaaaaagttgttgacttaaaagaagctaaatgttatagtaataaa

SEQ ID NO: 23 - DNA, 2985 bp, Bacillus licheniformis
  source 1..2985: mol_type = other DNA
acagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtc

SEQ ID NO: 24 - DNA, 53 bp, synthetic construct
  source 1..53: mol_type = other DNA
catagtaccgagaaactagaggatcctgggagctgatagcacagcgatttccc

SEQ ID NO: 25 - DNA, 50 bp, synthetic construct
  source 1..50: mol_type = other DNA
gtcgtaatgatttttataatgacggacggcttatggtatcacctagtgac

SEQ ID NO: 26 - DNA, 21 bp, synthetic construct
  source 1..21: mol_type = other DNA
gtcaagctggatctgccgctg

SEQ ID NO: 27 - DNA, 19 bp, synthetic construct
  source 1..19: mol_type = other DNA
ctgttctatgcatgtcatg

SEQ ID NO: 28 - DNA, 1904 bp, synthetic construct
  source 1..1904: mol_type = other DNA
gtcaagctggatctgccgctgaaatcgaaagtcaaacaataaaaaaatggagattccctaagaggggggtctccatttttaattcaagctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaatacattacattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttcaagcacgaaaaacacttcccggtgatcgggaggtgttttttgttaaaaagatcatgacatgcatagaacag

SEQ ID NO: 29 - DNA, 20 bp, synthetic construct
  source 1..20: mol_type = other DNA
catcccggatatcgtgtctg

SEQ ID NO: 30 - DNA, 21 bp, synthetic construct
  source 1..21: mol_type = other DNA
cttgagatcagggagaattcc

SEQ ID NO: 31 - DNA, 1864 bp, synthetic construct
  source 1..1864: mol_type = other DNA
catcccggatatcgtgtctgttaaagccatcgaccttccttaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaatacattacattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttacagaagctgcggaacctgaaaagaattcctttcaggttccgttttttttaggaattctccctgatctcaag

SEQ ID NO: 32 - DNA, 9214 bp, synthetic construct
  source 1..9214: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaagatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 33 - DNA, 9303 bp, synthetic construct
  source 1..9303: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaacatcaaaaacattgctaaaaaagcgtcagccttaaccgttgctgcggcactgctggccggaggtgcgccgcaagcgagtgcggcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatgaaagcttctcgaggttaacagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtc

SEQ ID NO: 34 - DNA, 12438 bp, synthetic construct
  source 1..12438: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagcttctcgaggttaacagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacgcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 35 - protein, 484 aa, synthetic construct
HHNGTNGTMMQYFEWHLPNDGQHWNRLRNDAANLKNLGITAVWIPPAWKGTSQNDVGYGAYDLYDLGEFNQKGTIRTKYGTRSQLQSAIASLQNNGIQVYGDVVMNHKGGADGTEWVQAVEVNPSNRNQEVTGEYTIEAWTKFDFPGRGNTHSSFKWRWYHFDGTDWDQSRRLNNRIYKFTGKGWDWEVDTENGNYDYLMYADVDMDHPEVINELRRWGVWYTNTLNLDGFRIDAVKHIKYSFTRDWLNHVRSTTGKNNMFAVAEFWKNDLGAIENYLHKTNWNHSVFDVPLHYNLYNASKSGGNYDMRQILNGTVVSKHPMHAVTFVDNHDSQPAEALESFVEAWFKPLAYALILTREQGYPSVFYGDYYGIPTHGVAAMKGKIDPILEARQKYAYGTQHDYLDHHNIIGWTREGNSAHPNSGLATIMSDGPGGSKWMYVGRHKAGQVWRDITGNRTGTVTINADGWGNFSVNGGSVSIWVNK

SEQ ID NO: 36 - DNA, 9215 bp, synthetic construct
  source 1..9215: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagcttctcgaggttaacagaggacggatttcctgaaggaaatccgtttttttattttcaagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 37 - DNA, 12511 bp, synthetic construct
  source 1..12511: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaatcgctgataaacagctgacatcaactaaaagcttcattaaatactttgaaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagagcagagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtccgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 38 - DNA, 9288 bp, synthetic construct
  source 1..9288: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaatcgctgataaacagctgacatcaactaaaagcttcattaaatactttgaaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagagcagagaggacggatttcctgaaggaaatccgtttttttattttacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtc

SEQ ID NO: 39 - DNA, 93 bp, synthetic construct
  source 1..93: mol_type = other DNA
tcgctgataaacagctgacatcaactaaaagcttcattaaatactttgaaaaaagttgttgacttaaaagaagctaaatgttatagtaataaa

SEQ ID NO: 40 - DNA, 1905 bp, synthetic construct
  source 1..1905: mol_type = other DNA
gtcaagctggatctgccgctgaaatcgaaagtcaaacaataaaaaaatggagattccctaagaggggggtctccatttttaattcaagtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagcttctcgaggttaacagaggacggatttcctgaaggaaatccgtttttttattttcaagcacgaaaaacacttcccggtgatcgggaggtgttttttgttaaaaagatcatgacatgcatagaacag

SEQ ID NO: 41 - DNA, 1849 bp, synthetic construct
  source 1..1849: mol_type = other DNA
catcccggatatcgtgtctgttaaagccatcgaccttccttaatcgctgataaacagctgacatcaactaaaagcttcattaaatactttgaaaaaagttgttgacttaaaagaagctaaatgttatagtaataaaacagaatagtcttttaagtaagtctactctgaatttttttaaaaggagagggtaaagatgaaaatgttgaaaaaggctgtgttgatagccgctgttttcttgctggccgcatttgccgggagtacagaagccagtgctcaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagagcagagaggacggatttcctgaaggaaatccgtttttttattttacagaagctgcggaacctgaaaagaattcctttcaggttccgttttttttaggaattctccctgatctcaag

SEQ ID NO: 42 - DNA, 8133 bp, synthetic construct
  source 1..8133: mol_type = other DNA
atgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgagctctcgctgataaacagctgacatcaactaaaagcttcattaaatactttgaaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcacaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgaaagagcagagaggacggatttcctgaaggaaatccgtttttttattttgcggccgcatattccgcattcgcaatgcctaccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaaacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattct

SEQ ID NO: 43 - DNA, 9176 bp, synthetic construct
  source 1..9176: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcagagctcgtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcacaccacaacggaacaaacggcacaatgatgcagtattttgaatggcacctgccgaatgatggacagcattggaatagactgagaaacgacgcagcgaacctgaagaaccttggcatcaacgcagtctggattccgcctgcgtggaaaggcacatcacaaaatgatgtcggctatggcgcatatgacctgtacgacctgggagagttcaaccagaagggaacaatcagaacgaaatatggaacaagatcacaactgcaaagcgctatcgcgcgcctgcaaaataatggcatccaagtttttggcgacgtggtcatgaaccacaaaggcggagcagacggaacggaacgcgttcaagcggtcgaggtgaatccgagcaacagaaaccaagaggttacgggcgaatacacgatcgaagcctggacaaagttcgactttccgggcagaggcaatacacactctagcttcaagtggagatggtatcactttgacggcacggactgggatcaaagcagaaacctgaataacagaatctataagtttacaggcaaagcatgggattgggaggtggacacagaaaacggaaactatgactatctgatgtatgctgacgtcgacatggatcatccggaggtcatcaatgagctgagaagatggggcgtttggtacacgaacacactgaacctggatggattcagaattgacgcagtcaaacacatcaagtaccagtttacaagagactggcttaaccacgtgagatcaacaacgggaaagaacaatatgttcgccgttgcggagttttggaagaatgatctgggcgcaatcgagaactatcttagcaagacgaactggaatcatagcgtcttcgatgtcccgctgcactacaatctgtataatgcatcaaagtcaggcggcaactacgatatgagacaaatcctgaatggaacggtcgtctcaaaacacccgatccacgccgtcacgtttgtcgataatcacgattcacaaccggcagaagcccttgagagctttgttgaggcatggttcaaaccgctggcctatgctctgatcctgacaagagagcaaggctatccgtcagtgttttacggcgattactacggcattccgacacatggcgtcgcagccatgaaaggaaagattgatccgattctggaggctagacagaaatacgcctacggaacgcaacacgattaccttgatcaccataacatcatcggatggacgagagagggaaactcagcacatcctaactcaggccttgcaacaattatgtcagatggaccgggaggcagcaaatggatgtacgttggcagacacaaggcaggacaggtttggagagatatcacgggcaatagaacaggaacagttacaatcaacgctgacggctggggcaattttagcgttaatggcggctcagttagcatctgggtgaataaatgatagtaaaaagaagcaggttcctccatacctgcttctttttatttgtcagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggca

SEQ ID NO: 44 - DNA, 57 bp, synthetic construct
  source 1..57: mol_type = other DNA
acagaatagtcttttaagtaagtctactctgaatttttttaaaaggagagggtaaag

SEQ ID NO: 45 - DNA, 87 bp, synthetic construct
  source 1..87: mol_type = other DNA
atgaaacaacaaaaacggctttacgcccgattgctgacgctgttatttgcgctcatcttcttgctgcctcattctgcagcttcagca

SEQ ID NO: 46 - DNA, 81 bp, synthetic construct
  source 1..81: mol_type = other DNA
gtgaaaatgttgaaaaaggctgtgttgatagccgctgttttcttgctggccgcatttgccgggagtacagaagccagcgct

SEQ ID NO: 47 - DNA, 2723 bp, Bacillus licheniformis
  source 1..2723: mol_type = other DNA
cctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccg

SEQ ID NO: 48 - DNA, 3602 bp, Bacillus licheniformis
  source 1..3602: mol_type = other DNA
gcggccgcatattccgcattcgcaatgcctaccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaaacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcag

(19) United States
(12) Patent Application Publication
(10) Pub. No.: US 2024/0360430 A1
(43) Pub. Date: Oct. 31, 2024

(54) METHODS AND COMPOSITIONS FOR ENHANCED PROTEIN PRODUCTION IN BACILLUS CELLS

(71) Applicant: DANISCO US INC., Palo Alto, CA (US)

(72) Inventors: Ryan L. Frisch, Newark, DE (US); Wonchul Suh, Hockessin, DE (US); Chris Leeflang, Twisk (NL); Marc Kolkman, Oegstgeest (NL); Hongxian He, Wilmington, DE (US)

(21) Appl. No.: 18/683,746

(22) PCT Filed: Aug. 19, 2022
(86) PCT No.: PCT/US2022/075208

§ 371 Date: Feb. 14, 2024

Related U.S. Application Data
(60) Provisional application No. 63/235,476, filed on Aug. 20, 2021.

(51) Int. Cl. C12N 9/28; C12N 1/20; C12N 15/75; C12R 1/07

(52) U.S. Cl. CPC C12N 9/2417; C12N 1/205; C12N 15/75; C12R 2001/07

(57) ABSTRACT

The present disclosure is generally related to recombinant microbial cells expressing heterologous proteins of interest. Certain aspects of the disclosure are therefore related to, inter alia, recombinant Bacillus cells having enhanced protein production capabilities, novel protein signal sequences, recombinant polynucleotides encoding heterologous proteins of interest, and related compositions and/or methods thereof. Thus, as exemplified herein, the recombinant Bacillus cells of the instant disclosure are particularly suitable for use in the expression, production and secretion of heterologous proteins.

### FIELD

The present disclosure is generally related to the fields of microbial cells, molecular biology, fermentation, protein production, and the like. Certain aspects of the disclosure are related to, inter alia, recombinant Bacillus cells having enhanced protein production capabilities.

### REFERENCE TO A SEQUENCE LISTING

The contents of the electronic submission of the text file Sequence Listing, named “NB41810-US-PSP_SequenceListing.txt” was created on Aug. 15, 2022 and is 192 KB in size, which is hereby incorporated by reference in its entirety.

### BACKGROUND

Gram-positive bacteria such as Bacillus subtilis, Bacillus licheniformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, Bacillus sp. cells are well known for their production of amylases (Jensen et al., 2000; Raul et al., 2014) and proteases (Brode et al., 1996) necessary for food, textile, laundry, medical instrument cleaning, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010). Thus, the production of proteins (e.g., enzymes, antibodies, receptors, etc.) in Gram-positive bacterial cells is an area of high interest in the biotechnological arts, wherein small improvements in protein yield are quite significant when the protein is produced in large industrial quantities.

As described hereinafter, the instant disclosure is related to the highly desirable and unmet needs for obtaining, constructing, producing and the like, Gram-positive host cells having increased protein production capabilities. More specifically, certain aspects of the instant disclosure are related to, among other things, compositions and methods for constructing recombinant Bacillus cells having enhanced protein production capabilities, which recombinant cells are particularly useful in the production of heterologous proteins.

### SUMMARY

As briefly set forth above, certain aspects of the disclosure are related to recombinant Bacillus cells (strains) having enhanced protein production phenotypes. Thus, in certain aspects, the disclosure provides, among other things, nucleic acids encoding novel protein signal sequences and polynucleotide constructs (e.g., vectors, expression cassettes) thereof which are particularly suitable for use in constructing recombinant (modified) Bacillus host cells having increased protein production capabilities described herein.

More particularly, in certain embodiments, a nucleic acid of the disclosure encodes a modified Bacillus licheniformis SacB signal sequence (modSacBss) comprising SEQ ID NO: 2. In certain other embodiments, a nucleic acid of the disclosure encodes a modified Bacillus licheniformis Bli03445 signal sequence (modBli03445ss) comprising SEQ ID NO: 6.

In other embodiments, a recombinant polynucleotide of the disclosure comprises an upstream (5′) nucleic acid encoding a signal sequence comprising SEQ ID NO: 2 operably linked to a downstream (3′) nucleic acid encoding a protein of interest (POI). In certain other embodiments, a recombinant polynucleotide of the disclosure comprises an upstream (5′) nucleic acid encoding a signal sequence comprising SEQ ID NO: 6 operably linked to a downstream (3′) nucleic acid encoding a protein of interest (POI).

In other aspects, a recombinant polynucleotide of the disclosure comprises an upstream (5′) promoter sequence operably linked to a downstream nucleic acid encoding a signal sequence comprising SEQ ID NO: 2 operably linked to a downstream (3′) nucleic acid encoding a protein of interest (POI). In certain other embodiments, a recombinant polynucleotide of the disclosure polynucleotide comprises an upstream (5′) promoter sequence operably linked to a downstream nucleic acid encoding a signal sequence comprising SEQ ID NO: 6 operably linked to a downstream (3′) nucleic acid encoding a protein of interest (POI). In other embodiments, the recombinant polynucleotide further comprises a terminator sequence downstream (3′) and operably linked to the nucleic acid encoding the POI.

Thus, certain other embodiments provide recombinant Bacillus cells comprising at least one introduced polynucleotide set forth herein. In other aspects, the recombinant Bacillus cells of the disclosure comprise at least two introduced polynucleotides set forth herein. In certain other aspects, the recombinant Bacillus cells of the disclosure are rendered deficient in the production of one or more native (endogenous) genes. In certain embodiments, Bacillus cells are rendered deficient in the production of one or more native (endogenous) proteases.

Other embodiments of the disclosure are related to recombinant Bacillus cells expressing an introduced polynucleotide encoding a heterologous protein of interest (POI), wherein the polynucleotide comprises an upstream (5′) nucleic acid encoding a signal sequence comprising SEQ ID NO: 2 operably linked to a downstream (3′) nucleic acid encoding the POI. In other embodiments, the disclosure is related to recombinant Bacillus cells expressing at least two introduced polynucleotides encoding a heterologous protein of interest (POI), wherein the introduced polynucleotides comprises an upstream (5′) nucleic acid encoding a signal sequence comprising SEQ ID NO: 2 operably linked to a downstream (3′) nucleic acid encoding the POI.

In other embodiments, the disclosure is related to recombinant Bacillus cells expressing an introduced polynucleotide encoding a heterologous protein of interest (POI), wherein the polynucleotide comprises an upstream (5′) nucleic acid encoding a signal sequence comprising SEQ ID NO: 6 operably linked to a downstream (3′) nucleic acid encoding the POI. In certain other embodiments, the disclosure is related to recombinant Bacillus cells expressing at least two introduced polynucleotides encoding a heterologous protein of interest (POI), wherein the introduced polynucleotides comprises an upstream (5′) nucleic acid encoding a signal sequence comprising SEQ ID NO: 6 operably linked to a downstream (3′) nucleic acid encoding the POI.

Thus, certain other embodiments are related to recombinant Bacillus cells expressing at least two introduced polynucleotides encoding a heterologous protein of interest (POI), wherein the first and second introduced polynucleotides comprise an upstream (5′) nucleic acid encoding a signal sequence comprising SEQ ID NO: 2 operably linked to a downstream (3′) nucleic acid encoding the POI, and an upstream (5′) nucleic acid encoding a signal sequence comprising SEQ ID NO: 6 operably linked to a downstream (3′) nucleic acid encoding the POI. respectively.

In other embodiments, the disclosure relates to methods for expressing heterologous proteins of interest in Bacillus host cells. More particularly, certain embodiments are related to methods for expressing a heterologous protein of interest (POI) in a Bacillus cell comprising obtaining or constructing a Bacillus cell comprising an introduced polynucleotide comprising an upstream (5′) promoter sequence operably linked to a downstream nucleic acid encoding a signal sequence comprising SEQ ID NO: 2 operably linked to a downstream (3′) nucleic acid encoding the POI, and fermenting the Bacillus cell under suitable conditions for the expression of the POI. Certain other embodiments are related to methods for expressing a heterologous protein of interest (POI) in a Bacillus cell comprising obtaining or constructing a Bacillus cell comprising an introduced polynucleotide comprising an upstream (5′) promoter sequence operably linked to a downstream nucleic acid encoding a signal sequence comprising SEQ ID NO: 6 operably linked to a downstream (3′) nucleic acid encoding the POI, and fermenting the Bacillus cell under suitable conditions for the expression of the POI.

In certain aspects of the methods, the Bacillus cell secretes the POI into the fermentation broth when fermented under suitable conditions for the expression of the POI.

### BRIEF DESCRIPTION OF DRAWINGS

FIG. 1 shows the amino acid sequence of a native B. licheniformis SacB (protein) signal sequence (FIG. 1A—SacBss; SEQ ID NO: 1) and a modified SacB signal sequence (FIG. 1B—modSacBss; SEQ ID NO: 2). As presented in FIG. 1, the amino acid positions of a particular protein signal sequence may be described according to the primary amino acid sequence (FIG. 1A and FIG. 1B; see 1^{st} sequence), described and numbered from the amino-terminus (NH₂; FIG. 1A and FIG. 1B; see 2^{nd} sequence), and/or described and numbered according to the cleavage site of a particular signal sequence (FIG. 1A and FIG. 1B; see 3^{rd} sequence). FIG. 1C presents an alignment of the native (SacBss) and modified (modSacBss) signal sequences.

FIG. 2 shows the amino acid sequence of a native B. licheniformis AmyLss signal sequence (FIG. 2A, AmyLss; SEQ ID NO: 3) and a modified modAmyLss signal sequence (FIG. 2B, modAmyLss; SEQ ID NO: 4). FIG. 2C shown an alignment of the native (AmyLss) and modified (modAmyLss) signal sequences.

FIG. 3 shows the amino acid sequence of a native B. licheniformis Bli03445 signal sequence (FIG. 3A, Bli03445; SEQ ID NO: 5) and a modified Bli03445 signal sequence (FIG. 3B, modBli03445; SEQ ID NO: 6). FIG. 3C shown an alignment of the native (Bli03445) and modified (modBli03445) signal sequences.

### BRIEF DESCRIPTION OF THE BIOLOGICAL SEQUENCES

SEQ ID NO: 1 is an amino acid sequence of the native B. licheniformis SacB signal sequence (SacBss).

SEQ ID NO: 2 is an amino acid sequence of sequence of a modified B. licheniformis SacB signal sequence (modSacBss).

SEQ ID NO: 3 is an amino acid sequence of the native B. licheniformis AmyL signal sequence (AmyLss).

SEQ ID NO: 4 is an amino acid sequence of sequence of a modified AmyL B signal sequence (modAmyLss).

SEQ ID NO: 5 is an amino acid sequence of the native B. licheniformis Bli03445 signal sequence (Bli03445ss).

SEQ ID NO: 6 is an amino acid sequence of sequence of a modified Bli03445 signal sequence (modBli03445ss).

SEQ ID NO: 7 is a synthetic DNA sequence of pWS733.

SEQ ID NO: 8 is the amino acid sequence of the Amylase 1 reporter protein.

SEQ ID NO: 9 is a synthetic DNA sequence of the 1^{st} Amylase 1 cassette (modSacBss; [pro-modSacBss-amylase 1] lysA).

SEQ ID NO: 10 is a B. licheniformis lysA upstream (lysA.up) sequence.

SEQ ID NO: 11 is a B. licheniformis lysA open reading frame (ORF).

SEQ ID NO: 12 is a synthetic p2 promoter.

SEQ ID NO: 13 is a B. subtilis aprE 5′-UTR sequence.

SEQ ID NO: 14 is a B. licheniformis amyL terminator sequence.

SEQ ID NO: 15 is a B. licheniformis lysA downstream (lysA.down) sequence.

SEQ ID NO: 16 is a synthetic primer sequence named “ws683”.

SEQ ID NO: 17 is a synthetic primer sequence named “ws688”.

SEQ ID NO: 18 is a synthetic DNA sequence of pWS735.

SEQ ID NO: 19 is a synthetic DNA sequence of the 2^{nd} Amylase 1 cassette (modSacBss; [pro-modSacBss-amylase 1] serA).

SEQ ID NO: 20 is a B. licheniformis serA upstream (serA.up) sequence.

SEQ ID NO: 21 is a B. licheniformis serA ORF.

SEQ ID NO: 22 is a synthetic p3 promoter.

SEQ ID NO: 23 is a B. licheniformis serA downstream (serA.down) sequence.

SEQ ID NO: 24 is a synthetic primer sequence named “ws709”.

SEQ ID NO: 25 is a synthetic primer sequence named “ws714”.

SEQ ID NO: 26 is a synthetic primer sequence named “ws775”.

SEQ ID NO: 27 is a synthetic primer sequence named “ws776”.

SEQ ID NO: 28 is a synthetic 1904 bp DNA fragment for screening integration of the 1^{st} Amylase 1 (modSacBss) cassette.

SEQ ID NO: 29 is a synthetic primer sequence named “1617”.

SEQ ID NO: 30 is a synthetic primer sequence named “ws717”.

SEQ ID NO: 31 is a synthetic 1864 bp fragment for screening integration of the 2^{nd} Amylase 1 (modSacBss) cassette.

SEQ ID NO: 32 is a synthetic DNA sequence of a 1^{st} Amylase 1 cassette (modAmyLss; pro-modAmyLss-amylase 1] lysA).

SEQ ID NO: 33 is a synthetic DNA sequence of a 2^{nd} Amylase 1 cassette (modAmyLss; pro-modAmyLss-amylase 1] serA).

SEQ ID NO: 34 is a synthetic DNA sequence of pWS743.

SEQ ID NO: 35 is the amino acid sequence of the Amylase 2 reporter protein.

SEQ ID NO: 36 is a synthetic DNA sequence of a 1 Amylase 2 cassette (modBli03445ss; [pro-modBli03445ss-amylase 2] lysA).

SEQ ID NO: 37 is a synthetic DNA sequence of pWS745.

SEQ ID NO: 38 is a synthetic DNA sequence of a 2^{nd} Amylase 2 cassette (modBli03445ss; [pro-modBli03445ss-amylase 2] serA).

SEQ ID NO: 39 is a synthetic p1 promoter.

SEQ ID NO: 40 is a synthetic 1905 bp fragment for screening integration of the 1^{st} Amylase 2 cassette (modBli03445ss; [pro-modBli03445ss-amylase 2] lysA).

SEQ ID NO: 41 is a synthetic 1849 bp fragment for screening integration of the 2^{nd} Amylase 2 cassette (modBli03445ss; [pro-modBli03445ss-amylase 2] serA).

SEQ ID NO: 42 is a synthetic DNA sequence of a 1^{st} Amylase 2 cassette (modAmyLss; [pro-modAmyLss-amylase 2] serA).

SEQ ID NO: 43 is a synthetic DNA sequence of a 2^{nd} Amylase 2 cassette (modAmyLss; [pro-modAmyLss-amylase 2] lysA).

SEQ ID NO: 44 is a synthetic DNA sequence of a modified B. subtilis aprE 5′-UTR sequence.

SEQ ID NO: 45 is a synthetic DNA sequence (modAmyLss) of a modified B. licheniformis AmyL signal sequence (modAmyLss).

SEQ ID NO: 46 is a synthetic DNA sequence (modBli03445ss) of the modified B. licheniformis Bli03445 signal sequence (mod Bli03445ss).

SEQ ID NO: 47 is B. licheniformis serA upstream (serA.up) sequence.

SEQ ID NO: 48 is a serA cassette and downstream homology arm to the serA cassette.

### DETAILED DESCRIPTION

### I. Overview

As described herein, the instant disclosure addresses numerous ongoing and unmet needs in the art, particularly as related to the industrial scale production recombinant proteins. Certain embodiments of the instant disclosure provide, among other things, recombinant Bacillus cells capable expressing increased amounts of proteins of interest. Certain aspects of the disclosure therefore provide, among other things, novel (recombinant) Bacillus cells comprising introduced nucleic acids (e.g., vectors, expression cassettes) encoding proteins of interest, polynucleotide constructs encoding modified (protein) signal sequences operably linked to a downstream nucleic acid a encoding protein of interest, recombinant Bacillus cells comprising one or more introduced polynucleotide constructs, and related methods for cultivating and expressing heterologous proteins of interest in a recombinant Bacillus cell of the disclosure and the like.

### II. Definitions

In view of the recombinant cells, nucleic acids, polynucleotides, proteins of interest and the like, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely”, “only”, “excluding”, “not including” and the like, in connection with the recitation of claim elements, or use of a “negative” limitation or proviso thereof. An example of a proviso used herein, in certain embodiments, a “recombinant lectin protein” produced and/or purified according to the instant disclosure is not a “His-tagged lectin”.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a Gram-positive cell) that has been altered such that the expression of a heterologous nucleic acid molecule or an endogenous nucleic acid molecule or a gene can be controlled. Recombinant also refers to a cell that is derived from a non-natural cell, or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules (e.g., polynucleotide expression constructs) that are not found in identical or homologous form within a native (wild-type) cell, or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric DNA sequence that would not otherwise be found in the genome.

The term “derived” encompasses the terms “originated”, “obtained”, “obtainable”, and “created” and generally indicates that one specified material or composition finds its origin in another specified material or composition, or has features that can be described with reference to the other specified material or composition. For example, recombinant Gram-positive bacterial cells of the disclosure may be derived/obtained from any known Gram-positive bacterial strains.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, an “endogenous gene” refers to a gene in its natural location in the genome of an organism.

As used herein, a “heterologous” gene, a “non-endogenous” gene, or a “foreign” gene refer to a gene not normally found in the host organism, but that is introduced into the host organism by gene transfer. The term “foreign” gene(s) comprises native genes inserted into a non-native organism and/or chimeric genes inserted into a native or non-native organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase during translocation.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters can be constitutive promoters, inducible promoters, tunable promoters, hybrid promoters, synthetic promoters, tandem promoters, etc. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, “a functional promoter sequence controlling the expression of a gene of interest linked to the gene of interest's protein coding sequence” refers to a promoter sequence which controls the transcription and translation of the coding sequence in a desired Gram-positive host cell. For example, in certain embodiments, the present disclosure is directed to a polynucleotide comprising an upstream (5′) promoter (or 5′ promoter region, or tandem 5′ promoters and the like) functional in a Gram-positive cell, wherein the promoter region is operably linked to a nucleic acid sequence encoding a protein of interest.

The term “operably linked” as used herein refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal sequence), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, transcription leader sequences, RNA processing site, effector binding site and stem-loop structures.

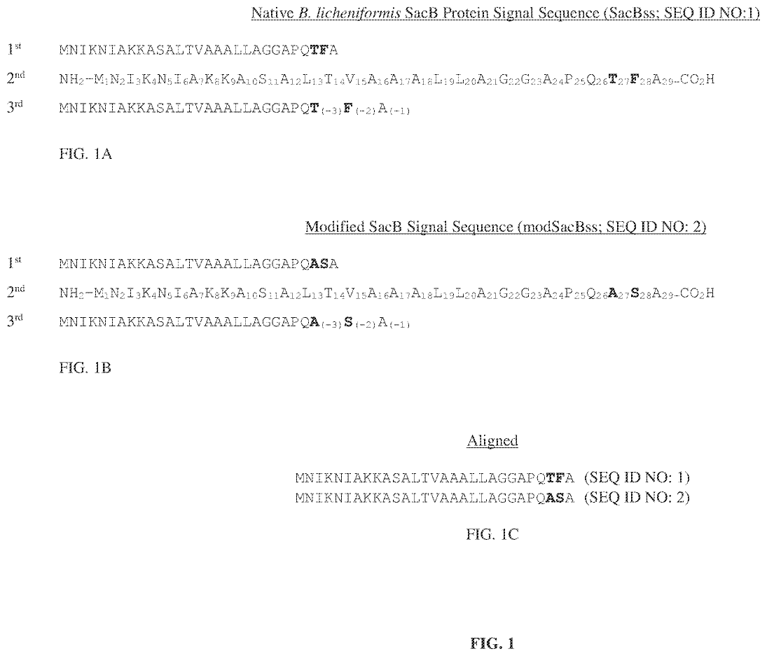

As used herein, a native B. licheniformis “SacB (protein) signal sequence” (abbreviated “SacBss”) comprises the amino acid sequence of SEQ ID NO:1, as shown in FIG. 1.

As used herein, a modified “SacB signal sequence” (abbreviated “modSacBss”) does not comprise the amino acid sequence of SEQ ID NO: 1. In certain embodiments, a modSacBss comprises an amino acid substitution at amino acid position 27, and/or comprises an amino acid substitution at amino acid position 28, as shown in FIG. 1. In certain aspects, a modSacBss comprises a threonine (T) to alanine (A) substation at position 27 and/or a phenylalanine (F) to serine (S) substation at position 28. In certain embodiments, a modSacBss comprises a T to A substation at position 27 and a F to S substation at position 28, as shown in SEQ ID NO: 2.

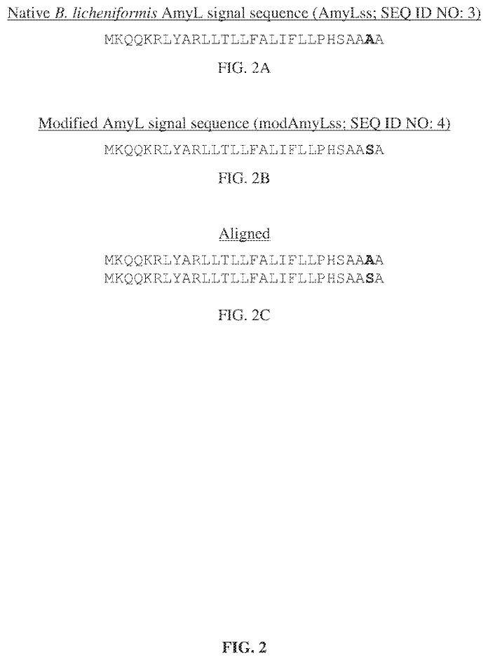

As used herein, a native B. licheniformis “AmyL (protein) signal sequence” (abbreviated, “AmyLss”) comprises the amino acid sequence of SEQ ID NO: 3, as shown in FIG. 2.

As used herein, a modified B. licheniformis “AmyL (protein) signal sequence” (abbreviated, “modAmyLss”) comprises the amino acid sequence of SEQ ID NO: 4, as shown in FIG. 2.

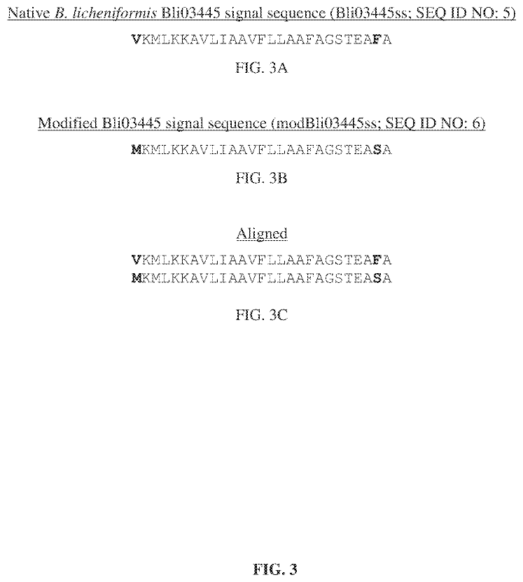

As used herein, a native B. licheniformis “Bli03445 (protein) signal sequence” (abbreviated, “Bli03445ss”) comprises the amino acid sequence of SEQ ID NO: 5, as shown in FIG. 3.

As used herein, a modified “Bli03445 signal sequence” (abbreviated, “modBli03445ss”) does not comprise the amino acid sequence of SEQ ID NO: 5. In certain embodiments, a modBli03445ss comprises an amino acid substitution at amino acid position 1, and/or comprises an amino acid substitution at amino acid position 26, as shown in FIG. 3. In certain aspects, a modBli03445ss comprises a valine (V) to methionine (M) substitution at amino acid position 1, and/or comprises a phenylalanine (F) to serine (S) substitution at amino acid position 26, as shown in SEQ ID NO: 6.

As used herein, the terms “Amylase 1^{st} (abbreviated “Amy1”) and/or “Amylase 2” (abbreviated “Amy2”) are not meant to be limiting, but rather refer to exemplary amylase reporter proteins of the disclosure. For example, in certain aspects, the disclosure demonstrates enhanced expression of such exemplary amylase reporters, e.g., Amy1 (SEQ ID NO: 8); Amy2 (SEQ ID NO: 35). As described and contemplated herein, any suitable protein of interest (e.g., enzymes) may be produced according to the recombinant strains and related methods of the disclosure.

As used herein, phrases such as first (1^{st}) copy of an “Amylase 1 modSacBss cassette” or 1^{st}” copy “Amylase 1 modSacBss” refer to an exemplary expression cassette (1^{st} Amylase 1 cassette; SEQ ID NO: 9) encoding Amylase 1. More particularly, the DNA sequence of the 1^{st} Amylase 1 (modSacBss) cassette comprises an upstream (5′) promoter (pro) sequence operably linked to the DNA sequence (modSacBss) encoding the modified SacB signal sequence (modSacBss) operably to the DNA sequence encoding the mature Amylase 1 protein. In certain aspects, the “1^{st} copy Amylase 1 modSacBss” cassette may be abbreviated as 1^{st} copy Amy1 “[pro-modSacBss-amylase 1]”.

As used herein, phrases such as second (2^{nd}) copy of “Amylase 1 modSacBss cassette” or 2^{nd} copy “Amylase 1 modSacBss” refer to an exemplary expression cassette (2^{nd} Amylase 1 cassette; SEQ ID NO: 19) encoding Amylase 1. More particularly, the DNA sequence of the 2^{nd} Amylase 1 (modSacBss) cassette comprises an upstream (5′) promoter (pro) sequence operably linked to the DNA sequence (modSacBss) encoding the modified SacB signal sequence (modSacBss) operably to the DNA sequence encoding the mature Amylase 1 protein. In certain aspects, the “2^{nd} copy Amylase 1 modSacBss” cassette may be abbreviated as 2^{nd} copy Amy1 “[pro-modSacBss-amylase 1]”.

As used herein, phrases such as first (1^{st}) copy of an “Amylase 1 modAmyLss cassette” or 1^{st} copy “Amylase 1 modAmyLss” refer to an exemplary expression cassette (1^{st} Amylase 1 cassette; SEQ ID NO: 32) encoding Amylase 1. More particularly, the DNA sequence of the 1^{st} Amylase 1 (modAmyLss) cassette comprises an upstream (5′) promoter (pro) sequence operably linked to the DNA sequence (modAmyLss) encoding the modified AmyLss signal sequence (modAmyLss) operably to the DNA sequence encoding the mature Amylase 1 protein. In certain aspects, the “1^{st} copy Amylase 1 modAmyLss” cassette may be abbreviated as 1^{st} copy Amy1 “[pro-modAmyLss-amylase 1]”.

As used herein, phrases such as second (2^{nd}) copy of an “Amylase 1 modAmyLss cassette” or 2^{nd} copy “Amylase 1 modAmyLss” refer to an exemplary expression cassette (2^{nd} Amylase 1 cassette; SEQ ID NO: 33) encoding Amylase 1. More particularly, the DNA sequence of the 2^{nd} Amylase 1 (modAmyLss) cassette comprises an upstream (5′) promoter (pro) sequence operably linked to the DNA sequence (modAmyLss) encoding the modified AmyLss signal sequence (modAmyLss) operably to the DNA sequence encoding the mature Amylase 1 protein. In certain aspects, the “2^{nd} copy Amylase 1 modAmyLss” cassette may be abbreviated as 2^{nd} copy Amy1 “[pro-modAmyLss-amylase 1]”.

As used herein, phrases such as first (1^{st}) copy of “Amylase 2 modBli03445ss cassette” or 1^{st} copy “Amylase 2 modBli03445ss” refer to an exemplary expression cassette (1^{st} Amylase 2 cassette; SEQ ID NO: 36) encoding Amylase 2. More particularly, the DNA sequence of the 1^{st} Amylase 2 (modBli03445ss) cassette comprises an upstream (5′) promoter (pro) sequence operably linked to the DNA sequence (modBli03445ss) encoding the modified Bli03445 signal sequence (modBli03445ss) operably to the DNA sequence encoding the mature Amylase 2 protein. In certain aspects, the “1^{st} copy Amylase 2 modBli03445ss” cassette may be abbreviated as 1 copy Amy2 “[pro-modBli03445ss-amylase 2]”.

As used herein, phrases such as second (2^{nd}) copy of “Amylase 2 modBli03445ss cassette” or 2^{nd} copy “Amylase 2 modBli03445ss” refer to an exemplary expression cassette (2^{nd} Amylase 2 cassette; SEQ ID NO: 38) encoding Amylase 2. More particularly, the DNA sequence of the 2^{nd} Amylase 2 (modBli03445ss) cassette comprises an upstream (5′) promoter (pro) sequence operably linked to the DNA sequence (modBli03445ss) encoding the modified Bli03445 signal sequence (modBli03445ss) operably to the DNA sequence encoding the mature Amylase 2 protein. In certain aspects, the “2^{nd} copy Amylase 2 modBli03445ss” cassette may be abbreviated as 2^{nd} copy Amy2 “[pro-modBli03445ss-amylase 2]”.

As used herein, phrases such as first (1^{st}) copy of an “Amylase 2 modAmyLss cassette” or 1^{st} copy “Amylase 2 modAmyLss” refer to an exemplary expression cassette (1^{st} Amylase 2 cassette; SEQ ID NO: 42) encoding Amylase 2. More particularly, the DNA sequence of the 1^{st} Amylase 2 (modAmyLss) cassette comprises an upstream (5′) promoter (pro) sequence operably linked to the DNA sequence (modAmyLss) encoding the modified AmyLss signal sequence (modAmyLss) operably to the DNA sequence encoding the mature Amylase 2 protein. In certain aspects, the “1^{st} copy Amylase 2 modAmyLss” cassette may be abbreviated as 1^{st} copy Amy2 “[pro-modAmyLss-amylase 2]”.

As used herein, phrases such as second (2^{nd}) copy of an “Amylase 2 modAmyLss cassette” or 2^{nd} copy “Amylase 2 modAmyLss” refer to an exemplary expression cassette (2^{nd} Amylase 2 cassette; SEQ ID NO: 43) encoding Amylase 2. More particularly, the DNA sequence of the 2^{nd} Amylase 2 (modAmyLss) cassette comprises an upstream (5′) promoter (pro) sequence operably linked to the DNA sequence (modAmyLss) encoding the modified AmyLss signal sequence (modAmyLss) operably to the DNA sequence encoding the mature Amylase 2 protein. In certain aspects, the “2^{nd} copy Amylase 2 modAmyLss” cassette may be abbreviated as 2^{nd} copy Amy2 “[pro-modAmyLss-amylase 2]”.

As used herein, a parental B. licheniformis (host) strain named “BF619” comprises deletions of at least its endogenous (native) lysA (ΔlysA) and serA (ΔserA) genes. For example, in certain embodiments, a parental B. licheniformis strain comprises deletions of its native lysA (ΔlysA) and serA (ΔserA) genes, and may further comprise additional genetic modifications introduced therein. In certain aspects, recombinant B. licheniformis host cells may further a comprises a deletion or disruption of an endogenous (native) protease gene.

As used herein, the genus “Bacillus” includes all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, a “host cell” refers to a cell that has the capacity to act as a host or expression vehicle for a newly introduced DNA sequence. This, in certain embodiments of the disclosure, the host cells are Bacillus sp. or E. coli cells.

As used herein, a “modified cell” refers to a recombinant cell that comprises at least one genetic modification which is not present in the parental cell from which the modified (daughter) cell is derived.

As used herein, when the expression and/or production of a protein of interest (POI) in a recombinant (modified) cell is being compared to the expression and/or production of the same POI in an unmodified (control or parental) cell, it will be understood that the modified and unmodified cells are grown/cultivated/fermented under the same conditions (e.g., the same conditions such as media, temperature, pH and the like).

As used herein, an “increased amount”, when used in phrases such as “a recombinant cell ‘expresses/produces an increased amount’ of a protein of interest relative to the unmodified (control or parental) cell”, particularly refers to an “increased amount” of a protein of interest (POI) expressed/produced in by the recombinant cell, which “increased amount” is always relative to the unmodified (control or parental) cells expressing/producing the same POI, wherein the modified and unmodified cells are grown/cultured/fermented under the same conditions.

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as amylase activity), or total extracellular protein produced as compared to the parental host cell.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “introducing”, as used in phrases such as “introducing into a bacterial cell” or “introducing into a B. licheniformis cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the Bacillus sp. chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a polypeptide of interest that is desired to be expressed in a modified B. licheniformis (daughter) host cell, wherein the POI is preferably expressed at increased levels (i.e., relative to the “unmodified” (parental) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest or an endogenous protein of interest relative to the parental cell. In particular embodiments, an increased amount of a protein of interest produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

### III. Signal Sequences for Improved Protein Secretion

Generally accepted methods of secreting heterologous proteins often rely on the use of native (protein) signal sequences of similar proteins (e.g., signal sequences from AmyL or AmyE for amylases, or AprE, NprE, or AprL for proteases), or signal sequences that are native to the heterologous protein sequence. For example, protein translation, secretion and folding can be consecutive processes and/or concurrent processes, wherein the protein's signal sequence plays a dynamic role in all three processes. More particularly, as appreciated by one of skill in the art, the use of sub-optimal signal sequences can present particular problems, including among other things, poor or insufficient translation, secretion and/or folding of the heterologous protein, wherein non-optimal pairings of signal sequences to mature protein sequences can lead to bottlenecks in production and secretion of the protein, leading to misfolded/inactive product and/or the induction of cellular stress responses further decreasing the productivity of the host cell.

As set forth herein, Applicant has designed and constructed recombinant B. licheniformis strains expressing exemplary reporter proteins (i.e., heterologous proteins of interest), wherein the mature (amino acid) sequences of the reporter proteins (e.g., Amy1, Amy2) have been operably linked to an upstream (N-terminal) protein (secretion) signal sequence of the disclosure. More particularly, as presented in the Examples below (see, Examples 1-4), and briefly described herein, in certain aspects, a first (1^{st}) copy of the Amylase 1 cassette (SEQ ID NO: 7) was integrated into the lysA locus (Example 1) and a second (2^{nd}) copy of the Amylase 1 cassette (SEQ ID NO: 19) was integrated into the serA locus (Example 2) of a parental B. licheniformis host strain comprising deletions of its native (endogenous) lysA (ΔlysA) and serA (ΔserA) genes (e.g., strain BF613; ΔlysA ΔserA).

As generally detailed in Examples 1 and 2, the 1^{st} copy Amylase 1 cassette (1^{st} Amy1 cassette; [pro-modSacBss-amylase 1] lysA; SEQ ID NO: 9) comprises a DNA sequence encoding a modified B. licheniformis SacB signal sequence (modSacBss; SEQ ID NO: 2) which is operably linked to a DNA sequence (amylase 1) encoding the amylase 1 reporter; and the 2^{nd} copy Amylase 1 cassette (2^{nd} Amy1 cassette; [pro-modSacBss-amylase 1] serA; SEQ ID NO: 19) comprises a DNA sequence encoding the modified SacB signal sequence (modSacBss; SEQ ID NO: 2) operably linked to a DNA sequence (amylase 1) encoding the amylase 1 reporter.

As shown in FIG. 1, the modified SacB signal sequence (modSacBss; SEQ ID NO: 2; FIG. 1B), relative to the native SacB signal sequence (SacBss; SEQ ID NO: 1; FIG. 1A), comprises substitutions of Thr (T) to Ala (A) at the 3 position and Phe (F) to Ser (S) at the ⁻2 position (i.e., relative to the signal peptidase cleavage site, e.g., FIG. 1A/FIG. 1B, 3^{rd} sequence presented).

As set forth in Example 3, an exemplary B. licheniformis strain was constructed (WS2806) which comprises the 1^{st} and 2^{nd} copy Amy1 cassettes integrated into the lysA and serA loci, respectively. As described in Example 4, the WS2806 strain (comprising 1^{st} and 2^{nd} copy Amy1 cassettes, modSacBss; SEQ ID NO: 2) were assayed for production of Amylase 1 compared to a control strain comprising the 1^{st} and 2^{nd} copy Amy1 cassettes integrated into the lysA and serA loci, respectively.

As set forth in Example 4, the 1^{st} copy Amy1 cassette integrated into the control strain (1^{st} Amy1 cassette; [pro-modAmyLss-amylase 1] lysA) comprises a DNA sequence encoding a modified B. licheniformis AmyLss signal sequence (modAmyLss; SEQ ID NO: 4; FIG. 2B) which is operably linked to a DNA sequence (amylase 1) encoding Amylase 1, and the 2^{nd} copy Amy1 cassette integrated into the control strain (2^{nd} Amy1 cassette; [pro-modAmyLss-amylase 1] serA) comprises a DNA sequence encoding the modified AmyLss signal sequence (modAmyLss; SEQ ID NO: 4) operably linked to a DNA sequence (amylase 1) encoding Amylase 1. As presented in TABLE 6 (Example 4), the relative improvement in production of the Amylase 1 reporter from the WS2806 strain (modSacBss), as compared to production of the same Amylase 1 reporter from the control strain (modAmyLss), was significantly enhanced (about 18%) when expressed and secreted with the modified SacB signal sequence (modSacBss; SEQ ID NO: 2).

As presented in the Examples below (see, Examples 5-8), and briefly described herein, in certain aspects, (Example 5) a first (1^{st}) copy of the Amy2 cassette (SEQ ID NO: 36) was integrated into the lysA locus, and (Example 6) a second (2^{nd}) copy of the Amy 2 cassette (SEQ ID NO: 38) was integrated into the serA locus of a parental B. licheniformis host strain (BF613; ΔlysA ΔserA) comprising deletions of its native lysA (ΔlysA) and serA (ΔserA) genes.

As generally detailed in Examples 5 and 6, the 1^{st} copy Amy2 cassette (1^{st} Amy2 cassette; [pro-modBli03445ss-amylase 2] lysA]); SEQ ID NO: 36) comprises a DNA sequence encoding a modified B. licheniformis Bli03445 signal sequence (modBli03445ss; SEQ ID NO: 6) which is operably linked to a DNA sequence (amylase 2) encoding the Amylase 2 reporter; and the 2^{nd} copy Amy2 cassette (2^{nd} Amy2 cassette; [pro-modBli03445ss-amylase 2] serA; SEQ ID NO: 38) comprises a DNA sequence encoding the modified Bli03445 signal sequence (modBli0344ss; SEQ ID NO: 6) operably linked to a DNA sequence (amylase 2) encoding the Amylase 2 reporter.

As shown in FIG. 3, the modified Bli03445 signal sequence (modBli03445ss; SEQ ID NO: 6; FIG. 3B), relative to the native Bli03445 signal sequence (Bli03445ss; SEQ ID NO: 5; FIG. 3A), comprises substitutions of valine (V) to methionine (M) at the start codon (⁻29 position) and phenylalanine (F) to serine (S) at the ⁻2 position (i.e., relative to the signal peptidase cleavage site (+1 amino acid position) of the mature protein of interest operably linked thereto.

As set forth in Example 7, an exemplary B. licheniformis strain was constructed (WS2835) which comprises the 1^{st} and 2^{nd} copy Amy2 cassettes integrated into the lysA and serA loci, respectively. As described in Example 8, the WS2835 strain (comprising 1^{st} and 2^{nd} copy Amy2 cassettes with modBli03445ss; SEQ ID NO: 6) were assayed for production of Amylase 2 compared to a control strain comprising the 1^{st} and 2^{nd} copy Amy2 cassettes (with modAmyLss) integrated into the lysA and serA loci, respectively. As presented in TABLE 9 (Example 8), the relative improvement in the production of the Amylase 2 reporter from the WS2835 strain (modBli03345ss), as compared to production of the same Amylase 2 reporter from the control strain (modAmyLss), was significantly enhanced (about 19%) when expressed and secreted with the modified Bli03345 signal sequence (modBli03345ss; SEQ ID NO: 6).

Thus, certain embodiments of the disclosure provide, among other things, nucleic acids, polynucleotides, vectors, expression cassettes, regulatory elements, and the like, suitable for use in constructing recombinant (modified) Bacillus host cells. Certain aspects are therefore related to polynucleotides (e.g., expression cassettes) comprising an upstream (5′) promoter (pro) sequence operably linked to a downstream nucleic acid sequence (ss) encoding a modified (protein) signal sequence operably linked to a downstream (3′) nucleic acid sequence (poi) encoding a protein of interest.

For example, a generic polynucleotide sequence encoding an amino (N) terminal signal sequence in operable combination with a mature protein of interest (POI) is shown below in Scheme 1:

5′-[ss]-[poi]-3′ Scheme 1:

wherein the nucleic acid (ss) sequence encoding the N-terminal signal sequence (SS) is upstream (5′) and operably linked to a nucleic acid (poi) sequence encoding a mature protein of interest (POI).

In certain embodiments, polynucleotide expression cassettes may be described generically as shown in Scheme 2:

5′-[pro]-[ss]-[poi]-3′ Scheme 2:

wherein the promoter (pro) sequence is upstream (5′) and operably linked to a nucleic acid (ss) sequence encoding the N-terminal signal sequence (SS), which is upstream (5′) and operably linked to a nucleic acid (poi) sequence encoding a protein of interest (POI). In certain other embodiments, a polynucleotide may further comprise a terminator (term) sequence downstream (3′) and operably linked to the nucleic acid (poi) sequence encoding the mature POI.

In certain other aspects, the disclosure is related to one or more nucleic acids encoding one or more modified signal sequences of the disclosure, such as the modified signal sequences set forth in FIG. 1 and/or FIG. 3.

In certain embodiments, the disclosure relates to polynucleotide constructs (e.g., Scheme 2) encoding a protein of interest (POI), wherein the signal sequence (ss) comprises a modified B. licheniformis SacB (protein) signal sequence, or a modified B. licheniformis Bli03445 (protein) signal sequence, operably linked to a nucleic acid (amy) sequence encoding a mature amylase protein (Amy), as generically shown in Scheme 3:

5′-[pro]-[Modss]-[amy]-3′ Scheme 3:

Thus, certain aspects of the disclosure provide recombinant B. licheniformis strains/cells comprising one or more introduced polynucleotide constructs (e.g., expression cassettes) encoding one or more mature amylases comprising a novel (modified) N-terminal signal sequence of the disclosure. More particularly, as exemplified hereinafter, Applicant has constructed exemplary B. licheniformis strains capable of secreting enhanced amounts of amylase proteins.

As presented in FIG. 1, the native B. licheniformis SacB protein signal sequence comprises a twenty-nine (29) amino acid residue sequence (FIG. 1A; SEQ ID NO: 1). For example, the amino acid positions of a particular protein signal sequence may be described and numbered from the amino-terminus (NH₂), as indicated in FIG. 1A (2^{nd} sequence), and FIG. 1B (2^{nd} sequence). Alternatively, the amino acid positions may be described and numbered according to the cleavage site of a particular signal sequence. For example, the most C-terminal amino acid position of the SacBss (SEQ ID NO: 1) and the modSacBss (SEQ ID NO: 2) signal sequence can be designated with a negative 1 (⁻1) amino acid (position), the amino acid position to its left a negative 2 (⁻2), etc., as indicated in FIG. 1A (3^{rd} sequence) and FIG. 1B (3^{rd} sequence). Likewise, other native and/or modified signal sequences (FIG. 2 and FIG. 3) of the disclosure may be designated with similar specificity.

### IV. Recombinant Polynucleotides and Molecular Biology

As generally set forth above and further described below in the Examples, certain embodiments of the disclosure are related to recombinant (modified) Bacillus cells capable of producing increased amounts of heterologous proteins of interest. Certain embodiments are therefore related to methods for constructing such recombinant Bacillus cells having increased protein production capabilities. In certain embodiments, one or more expression cassettes encoding a protein of interest are introduced into Bacillus cells of the disclosure. In exemplary embodiments, the cassettes are integrated into the genome of the cell. For example, in certain embodiments, expression cassettes encoding a protein of interest were integrated into the lysA locus and the serA locus of a parental B. licheniformis cell comprising deletions of its native lysA (ΔlysA) and serA (ΔserA) genes. Thus, certain embodiments are related to, among other things, nucleic acids, polynucleotides (e.g., vectors, expression cassettes), regulatory elements, and the like, suitable for use in constructing recombinant (modified) Bacillus host cells.

In certain other aspects, Bacillus cells of the disclosure are rendered deficient in the production of one or more native (endogenous) genes. In certain embodiments, Bacillus cells of the disclosure are rendered deficient in the production of one or more native (endogenous) proteases. For example, in certain embodiments, a host cell of the disclosure is a Bacillus licheniformis cell deficient in the production of one or more native proteases selected from the group consisting of wprA, nprE, mpr, aprL, bprE, htrA, vpr and ispA.

Accordingly, as presented in the Examples and generally described herein, recombinant cells of the disclosure may be constructed by one of skill using standard and routine recombinant DNA and molecular cloning techniques well known in the art. Methods for genetically modifying cells include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, modified cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a Bacillus cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene encoding a protein of interest can be edited or disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure are generally chosen so that they are functional in the Bacillus cells (e.g., B. licheniformis cells, B. subtilis cells and the like). For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is describe in Publication No. WO2002/14490.

### V. Fermenting Bacillus Cells for Production of a Protein of Interest

As generally described above, certain embodiments are related to compositions and methods for constructing and obtaining Bacillus cells having increased protein production phenotypes. Thus, certain embodiments are related to methods of producing proteins of interest in Bacillus cells by fermenting the cells in a suitable medium. Fermentation methods well known in the art can be applied to ferment Bacillus cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### VI. Proteins of Interest

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a modified Bacillus cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental) cell.

In certain embodiments, a modified Bacillus cell of the disclosure exhibits an increased specific productivity (Qp) of a POI relative the (unmodified) parental cell. For example, the detection of specific productivity (Qp) is a suitable method for evaluating protein production. The specific productivity (Qp) can be determined using the following equation:

$$“ Qp = gP / gDCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

There are various assays known to those of ordinary skill in the art for detecting and measuring activity of intracellularly and extracellularly expressed proteins.

### VII. Exemplary Embodiments

1. A nucleic acid encoding a modified SacB signal sequence (modSacBss) comprising SEQ ID NO: 2.

2. A nucleic acid encoding a modified Bli03445 signal sequence (modBli03445ss) comprising SEQ ID NO: 6.

3. A polynucleotide comprising an upstream (5′) nucleic acid encoding a signal sequence comprising SEQ ID NO: 2 operably linked to a downstream (3′) nucleic acid encoding a protein of interest (POI).

4. A polynucleotide comprising an upstream (5′) nucleic acid encoding a signal sequence comprising SEQ ID NO: 6 operably linked to a downstream (3′) nucleic acid encoding a protein of interest (POI).

5. A polynucleotide comprising an upstream (5′) promoter operably linked to a downstream nucleic acid encoding a signal sequence comprising SEQ ID NO: 2 operably linked to a downstream (3′) nucleic acid encoding a protein of interest (POI).

6. A polynucleotide comprising an upstream (5′) promoter sequence operably linked to a downstream nucleic acid encoding a signal sequence comprising SEQ ID NO: 6 operably linked to a downstream (3′) nucleic acid encoding a protein of interest (POI).

7. The polynucleotide of any one of embodiments 3-6, further comprising a terminator sequence downstream (3′) and operably linked to the nucleic acid encoding the POI.

8. The polynucleotide of any one of embodiments 3-7, wherein the nucleic acid encoding the POI encodes an enzyme.

9. The polynucleotide of embodiment 8, wherein the enzyme is a hydrolase.

10. The polynucleotide of embodiment 9, wherein the hydrolase is an amylase.

11. A recombinant Bacillus cell comprising at least one introduced polynucleotide of any one of embodiments 5-7.

12. A recombinant Bacillus cell comprising at least two introduced polynucleotides of any one of embodiments 5-7.

13. The recombinant Bacillus cell of embodiment 11 or embodiment 12, rendered deficient in the production of one or more native (endogenous) genes.

14. The recombinant Bacillus cell of embodiment 13, rendered deficient in the production of one or more native (endogenous) proteases.

15. A recombinant Bacillus cell expressing an introduced polynucleotide encoding a heterologous protein of interest (POI), wherein the polynucleotide comprises an upstream (5′) nucleic acid encoding a signal sequence comprising SEQ ID NO: 2 operably linked to a downstream (3′) nucleic acid encoding the POI.

16. A recombinant Bacillus cell expressing at least two introduced polynucleotides encoding a heterologous protein of interest (POI), wherein the introduced polynucleotides comprises an upstream (5′) nucleic acid encoding a signal sequence comprising SEQ ID NO: 2 operably linked to a downstream (3′) nucleic acid encoding the POI.

17. A recombinant Bacillus cell expressing an introduced polynucleotide encoding a heterologous protein of interest (POI), wherein the polynucleotide comprises an upstream (5′) nucleic acid encoding a signal sequence comprising SEQ ID NO: 6 operably linked to a downstream (3′) nucleic acid encoding the POI.

18. A recombinant Bacillus cell expressing at least two introduced polynucleotides encoding a heterologous protein of interest (POI), wherein the introduced polynucleotides comprises an upstream (5′) nucleic acid encoding a signal sequence comprising SEQ ID NO: 6 operably linked to a downstream (3′) nucleic acid encoding the POI.

18. A recombinant Bacillus cell expressing at least two introduced polynucleotides encoding a heterologous protein of interest (POI), wherein the first and second introduced polynucleotides comprise an upstream (5′) nucleic acid encoding a signal sequence comprising SEQ ID NO: 2 operably linked to a downstream (3′) nucleic acid encoding the POI, and an upstream (5′) nucleic acid encoding a signal sequence comprising SEQ ID NO: 6 operably linked to a downstream (3′) nucleic acid encoding the POI. respectively.

19. A method for expressing a heterologous protein of interest (POI) in a Bacillus cell comprising: (a) obtaining or constructing a Bacillus cell comprising an introduced polynucleotide comprising an upstream (5′) promoter sequence operably linked to a downstream nucleic acid encoding a modified SacB signal sequence (modSacBss) comprising SEQ ID NO: 2 operably linked to a downstream (3′) nucleic acid encoding the POI, and (b) fermenting the Bacillus cell under suitable conditions for the expression of the POI.

20. The method of embodiment 19, wherein the Bacillus cell expresses an increased amount of the heterologous POI relative to a control Bacillus cell expressing the same POI when fermented under the same conditions, wherein the control Bacillus cell comprises an introduced polynucleotide comprising an upstream (5′) promoter sequence operably linked to a downstream nucleic acid encoding a native SacB signal sequence (SacBss) comprising SEQ ID NO: 1 operably linked to a downstream (3′) nucleic acid encoding the same POI.

21. The method of embodiment 19, wherein the Bacillus cell expresses an increased amount of the POI relative to a control Bacillus cell expressing the same POI when fermented under the same conditions, wherein the control Bacillus cell comprises an introduced polynucleotide comprising an upstream (5′) promoter operably linked to a downstream nucleic acid encoding modified AmyL signal sequence (modAmyLss) comprising SEQ ID NO: 4 operably linked to a downstream (3′) nucleic acid encoding the same POI.

22. The method of embodiment 19, wherein the Bacillus cell secretes the POI into the fermentation broth when fermented under suitable conditions for the expression of the POI.

23. The method of embodiment 19, further comprising recovering the POI from the fermentation broth.

24. A protein preparation comprising the POI recovered according to embodiment 23.

25. A method for expressing a heterologous protein of interest (POI) in a Bacillus cell comprising: (a) obtaining or constructing a Bacillus cell comprising an introduced polynucleotide comprising an upstream (5′) promoter operably linked to a downstream nucleic acid encoding a modified Bli03445 signal sequence (modBli03445) comprising SEQ ID NO: 6 operably linked to a downstream (3′) nucleic acid encoding the POI, and (b) fermenting the Bacillus cell under suitable conditions for the expression of the POI.

26. The method of embodiment 25, wherein the Bacillus cell expresses an increased amount of the POI relative to a control Bacillus cell expressing the same POI when fermented under the same conditions, wherein the control Bacillus cell comprises an introduced polynucleotide comprising an upstream (5′) promoter operably linked to a downstream nucleic acid encoding a native Bli03445 signal sequence (Bli03445) comprising SEQ ID NO: 5 operably linked to a downstream (3′) nucleic acid encoding the same POI.

27. The method of embodiment 25, wherein the Bacillus cell expresses an increased amount of the POI relative to a control Bacillus cell expressing the same POI when fermented under the same conditions, wherein the control Bacillus cell comprises an introduced polynucleotide comprising an upstream (5′) promoter operably linked to a downstream nucleic acid encoding modified AmyL signal sequence (modAmyLss) comprising SEQ ID NO: 4 operably linked to a downstream (3′) nucleic acid encoding the same POI.

28. The method of embodiment 25, wherein the Bacillus cell secretes the POI into the fermentation broth when fermented under suitable conditions for the expression of the POI.

29. The method of embodiment 28, further comprising recovering the POI from the fermentation broth.

30. A protein preparation comprising the POI recovered according to claim 29.

### EXAMPLES

Certain aspects of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Construction of a Template Plasmid for the 1^{st} Amylase 1 Cassette

The instant example describes construction of a template plasmid pWS733 (SEQ ID NO: 7) for the integration of a first (1^{st}) copy of amylase 1 (SEQ ID NO: 8) expression cassette. For example, the 1^{st} copy of the amylase 1 expression cassette (1^{st} Amylase 1 cassette; lysA::[p2-modSacBss-amylase 1] lysA; SEQ ID NO: 9) comprises an upstream (5′) homology arm (up) for the lysA locus (lysA.up; SEQ ID NO: 10) operably linked to DNA encoding the lysA ORF (SEQ ID NO: 11) operably linked to a synthetic p2 promoter (pro; SEQ ID NO: 12) operably linked to DNA encoding a B. subtilis aprE 5′-UTR (SEQ ID NO: 30) operably linked to DNA encoding a modified B. licheniformis SacB signal sequence (modSacBss; SEQ ID NO: 2) operably linked to DNA encoding amylase 1 (amylase 1) operably linked to a B. licheniformis amyL transcriptional terminator (SEQ ID NO: 14) operably linked to a downstream (3′) homology arm (down) for the lysA locus (lysA.down; SEQ ID NO: 15). More particularly, the modified SacB signal sequence (modSacBss; SEQ ID NO: 2) of the 1^{st} copy amylase 1 cassette (FIG. 1B), relative to the native SacB signal sequence (SacBss; SEQ ID NO: 1; FIG. 1A), comprises substitutions of Thr (T) to Ala (A) at the 3 position and Phe (F) to Ser (S) at the ⁻2 position (i.e., relative to the signal peptidase cleavage site; FIG. 1.)

The DNA fragments were amplified using Q5 DNA polymerase per the manufacturer's instructions. The PCR products were purified using Zymo clean and concentrate 5 columns per manufacturer's instructions. The DNA fragments were assembled into a plasmid pRS426 purchased from ATCC (ATCC® 77107™) by using yeast gap-repair cloning method (Joska et al., 2014), generating pWS733 (SEQ ID NO: 7).

### Example 2

### Construction of a Template Plasmid for the 2^{nd} Amylase 1 Cassette

The present example describes construction of a template plasmid pWS735 (SEQ ID NO: 18) for the integration of a second (2^{nd}) copy of amylase 1 (SEQ ID NO: 8) expression cassette. The 2^{nd} copy of amylase 1 expression cassette (2^{nd} Amylase 1 cassette; serA::[p3-modSacBss-amylase 1] serA; SEQ ID NO: 19) comprises an upstream (5′) homology arm (up) to the serA locus (serA.up; SEQ ID NO: 20) operably linked to the serA ORF (SEQ ID NO: 21) operably linked to a synthetic p3 promoter (pro; SEQ ID NO: 22) operably linked to DNA encoding the B. subtilis aprE 5′UTR (SEQ ID NO: 13) operably linked to DNA encoding the modified B. licheniformis SacB signal sequence (modSacBss; SEQ ID NO: 2) operably linked to DNA encoding amylase 1 (SEQ ID NO: 8) operably linked to the B. licheniformis amyL transcriptional terminator (SEQ ID NO: 14) operably linked to the downstream (3′) homology arm (down) to the serA locus (serA.down; SEQ ID NO: 23).

The DNA fragments were amplified using Q5 DNA polymerase per the manufacturer's instructions. The PCR products were purified using Zymo clean and concentrate 5 columns per manufacturer's instructions. The DNA fragments A-B-partial C1 and partial C2 were assembled into a plasmid pRS426 purchased from ATCC (ATCC® 77107™) by using yeast gap-repair cloning method (Joska et al., 2014), generating pWS735 (SEQ ID NO: 18).

### Example 3

### Construction of Ws2806 Strain Expressing Amylase 1 with modSacBss

In the instant example, the expression cassettes constructed and described in the preceding examples were integrated into an exemplary B. licheniformis host strain. More particularly, an exemplary B. licheniformis host (WS2806) was constructed by integrating the 1^{st} and 2^{nd} copy Amylase 1 cassettes into a B. licheniformis parental strain named BF719. For example, the recombinant B. licheniformis WS2806 strain was constructed by integration of the 1^{st} Amylase 1 cassette (amylase 1; SEQ ID NO: 9) into the lysA locus, and then the 2^{nd} Amylase 1 cassette (amylase 1; SEQ ID NO: 19) into the serA locus in BF719 (ΔserA ΔlysA) as described below.

The 1^{st} Amylase 1 cassette (SEQ ID NO: 9) integration fragment was generated by PCR amplification from a plasmid template pWS733 (SEQ ID NO: 7) with the ws683 (SEQ ID NO: 16) and ws688 (SEQ ID NO: 17) primer pair. The 2^{nd} Amylase 1 cassette (amylase 1; SEQ ID NO: 19) integration fragment was generated by PCR amplification from a plasmid template pWS735 (SEQ ID NO: 18) with the ws709 (SEQ ID NO: 24) and ws714 (SEQ ID NO: 25) primer pair.

The Amylase 1 expression cassettes were transformed into BF719 (ΔserA ΔlysA) strain using the method as described PCT Publication No. WO2019/040412. Briefly, the BF719 competent cells were generated by growing the strain overnight in L broth containing one hundred (100) ppm spectinomycin at 37° C. with 250 RPM shaking. The culture was diluted the next day to OD₆₀₀ of 0.7 of fresh L broth containing one hundred (100) ppm spectinomycin. This new culture was grown for one (1) hour at 37° C., 250 RPM shaking. D-xylose was added to 0.1% w·v⁻¹. The culture was grown for an additional four (4) hours at 37° C. and 250 RPM shaking. The cells were harvested at 1700-g for seven (7) minutes, and used as competent cells for transformation.

One hundred (100) μl of BF719 competent cells were mixed with twenty (20) μl of the 1^{st} Amylase 1 cassette integration fragment (lysA::[p2-modSacBss-amylase 1] lysA). The cell/DNA mixture was incubated at 1200 RPM, 37° C. for one and a half (1.5) hours. The mixture was then plated on TSS agar plates containing eighty-eight (88) ppm serine and hundred (100) ppm spectinomycin. The inoculated plates were incubated at 37° C. for forty-eight (48) hours. Transformed colonies were screened by PCR amplification with the ws775 (SEQ ID NO: 26) and ws776 (SEQ ID NO: 27) primer pair. This PCR product, a 1904 bp fragment (SEQ ID NO: 28), was sequenced using the method of Sanger and the ws775 and ws776 primers. A colony with the correct integration of the cassette (lysA::[p2-modSacBss-amylase 1] lysA; SEQ ID NO: 9) was stored as strain WS2804.

The WS2804 competent cells were generated as described above. One hundred (100) μl of WS2804 competent cells were mixed with twenty (20) μl of the 2^{nd} Amylase 1 cassette (serA::[p3-modSacBss-amylase 1] serA) integration fragment. The cell/DNA mixture was incubated at 1200 RPM, 37° C. for one and a half (1.5) hours. The mixture was then plated on TSS agar plates. The inoculated plates were incubated at 37° C. for forty-eight (48) hours. Transformed colonies were screened by PCR amplification with the 1617 (SEQ ID NO: 29) and ws717 (SEQ ID NO: 30) primers pair. This PCR product, 1864 bp fragment (SEQ ID NO: 31), was sequenced using the method of Sanger and the 1617 (SEQ ID NO: 29) and ws717 (SEQ ID NO: 30) primer pair.

A colony with the correct integration of the 1^{st} Amylase 1 cassette (lysA::[p2-modSacBss-amylase 1] lysA; SEQ ID NO: 9) and the 2^{nd} Amylase 1 cassette (serA::[p3-modSacBss-amylase 1] serA; SEQ ID NO: 19) was passaged on L agar until the colonies were stored as strain WS2806 (serA::[p3-modSacBss-amylase 1] serA) lysA::[p2-modSacBss-amylase 1] lysA).

To test relative performance of the modified B. licheniformis SacB signal sequence (modSacBss; SEQ ID NO: 2) on amylase 1 production, the control strain with the modified B. licheniformis AmyL signal sequence (modAmyLss; SEQ ID NO: 4) was constructed in BF719 by the integration of a 1^{st} Amylase 1 cassette (lysA::[p2-modAmyLss-amylase 1] lysA; SEQ ID NO: 32) at the lysA locus and the integration of a 2^{nd} Amylase 1 cassette (serA::[p3-modAmyLss-amylase 1] serA; SEQ ID NO: 33) at the serA locus. The 1^{st} Amylase 1 cassette lysA::[p2-modAmyLss-amylase 1] lysA; SEQ ID NO: 32) comprises an upstream homology arm (up) for the lysA locus (lysA.up; SEQ ID NO: 10) operably linked to DNA sequence encoding lysA (ORF; SEQ ID NO: 11) operably linked to the synthetic p2 promoter (pro; SEQ ID NO: 12) operably linked to DNA encoding a B. subtilis aprE 5′-UTR (SEQ ID NO: 13) operably linked to DNA encoding a modified B. licheniformis AmyL signal sequence (modAmyLss; SEQ ID NO: 4) operably linked to DNA encoding amylase 1 operably linked to the B. licheniformis amyL transcriptional terminator (SEQ ID NO: 14) operably linked to the downstream homology arm (down) for the lysA locus (lysA.down; SEQ ID NO: 15). The 2^{nd} Amylase 1 cassette (serA::[p3-modAmyLss-amylase 1] serA; SEQ ID NO: 33) comprises an upstream homology arm (up) to the serA locus (serA.up; SEQ ID NO: 20) operably linked to serA ORF (SEQ ID NO: 21) operably linked to the synthetic p3 promoter (pro; SEQ ID NO: 22) operably linked to DNA encoding the modified B. subtilis aprE 5′-UTR (SEQ ID NO: 13) operably linked to DNA encoding the modified B. licheniformis AmyL signal sequence (modAmyLss; SEQ ID NO: 4) operably linked to DNA encoding amylase 1 (SEQ ID NO: 8) operably linked to the B. licheniformis amyL transcriptional terminator (SEQ ID NO: 14) operably linked to the downstream homology arm (down) to the serA locus (serA.down; SEQ ID NO: 23).

For example, the modified AmyL signal sequence (modAmyLss; SEQ ID NO: 4), relative to the native AmyL signal sequence (AmyLss; SEQ ID NO: 3), comprises substitutions of alanine (A) to serine (S) at the ⁻2 position, relative to the signal peptidase cleavage site (e.g., see FIG. 2).

### Example 4

### Effect of the Modified B. Licheniformis SacB Signal Sequence on Amylase 1 Production

In the present example, the WS2806 strain containing two (2) copies of amylase 1 expression cassettes with the modified B. licheniformis SacB signal sequence (modSacBss; SEQ ID NO: 2 and FIG. 1) were assayed for production of amylase 1 compared to the control BF822 strain containing two (2) copies of amylase 1 expression cassettes with the modified B. licheniformis AmyL signal sequence (modAmyLss; SEQ ID NO: 4 and FIG. 2) using standard small-scale conditions, as described in PCT Publication No. WO2018/156705 and WO2019/055261 (each incorporated herein by reference).

The Amylase 1 reporter protein production was quantified using the method of Bradford or the Ceralpha assay, wherein the relative improvement in production of amylase 1 from the WS2806 strain compared to the production for the BF822 (control) strain are presented below in TABLE 6.

TABLE 6
Relative Performance of modSacBss vs. modAmyLss
Signal Sequences on Amylase 1 Production

| | | | Relative Amy1 |
|---|---|---|---|
| Strain Name | Signal Sequence (ss) | SEQ ID NO | Production |
| WS2806 | modSacBss | 2 | 1.18 |
| BF822 | modAmyLss | 4 | 1.00 |

Thus, as shown in TABLE 6, the modified B. licheniformis SacB signal sequence (modSacBss) demonstrates an improvement in amylase 1 reporter protein production in the WS2806 strain relative to the B. licheniformis (control) strain BF822, comprising the modified B. licheniformis AmyL signal sequence (modAmyLss).

### Example 5

### Construction of a Template Plasmid for the 1^{st}_Amylase 2 Cassette

The instant example describes construction of a template plasmid pWS743 (SEQ ID NO: 34) for the integration of a first (1′) copy of amylase 2 (SEQ ID NO: 35) expression cassette. For example, the 1^{st} copy of the amylase 2 expression cassette (1^{st} Amylase 2 cassette; lysA::[p3-mod-Bli03445ss-amylase 2] lysA; SEQ ID NO: 36) comprises an upstream homology arm (up) to the lysA locus (lysA.up; SEQ ID NO: 10) operably linked to the lysA ORF (SEQ ID NO: 11) operably linked to the synthetic p3 promoter (pro; SEQ ID NO: 22) operably linked to DNA encoding a B. subtilis aprE 5′-UTR (SEQ ID NO: 13) operably linked to DNA encoding the modified B. licheniformis Bli03445 signal sequence (modBli03445ss; SEQ ID NO: 6) operably linked to DNA encoding amylase 2 operably linked to the B. licheniformis amyL transcriptional terminator (SEQ ID NO: 14) operably linked to a downstream (3′) homology arm (down) to the lysA locus (lysA.down; SEQ ID NO: 15).

More particularly, the modified B. licheniformis Bli03445 signal sequence (modBli03445ss; FIG. 3B) comprises a substitution of valine (V) to methionine (M) at the start codon (−29 position) and a substitution of phenylalanine (F) to serine (S) at the ⁻2 position (i.e., relative to the signal peptidase cleavage site of the mature Amylase 2 protein), relative to the native B. licheniformis Bli03445 signal sequence (Bli03445ss; FIG. 3A).

The DNA fragments were amplified using Q5 DNA polymerase per the manufacturer's instructions. The PCR products were purified using Zymo clean and concentrate 5 columns per manufacturer's instructions. The DNA fragments A, B and C were assembled into a plasmid pRS426 purchased from ATCC (ATCC® 77107™) by using yeast gap-repair cloning method (Joska et al., 2014) generating pWS743 (SEQ ID NO: 34).

### Example 6

### Construction of a Template Plasmid for the 2^{nd}_Amylase 2 Cassette

The instant example describes construction of a template plasmid pWS745 (SEQ ID NO: 37) for the integration of a second (2^{nd}) copy of Amylase 2 (SEQ ID NO: 35) expression cassette. For example, the 2^{nd} copy of Amylase 2 expression cassette (2^{nd} Amylase 2 cassette; serA::[p1-modBli03445ss-amylase 2] serA; SEQ ID NO: 38) comprises an (5′) upstream homology arm (up) to the serA locus (serA.up; SEQ ID NO: 20) operably linked to the serA ORF (SEQ ID NO: 21) operably linked to the synthetic p1 promoter (pro; SEQ ID NO: 39) operably linked to DNA encoding a B. subtilis aprE 5′-UTR (SEQ ID NO: 13) operably linked to DNA encoding the modified B. licheniformis Bli03445 signal sequence (modBli03445ss; SEQ ID NO: 6) operably linked to DNA encoding amylase 2 operably linked to the B. licheniformis amyL transcriptional terminator (SEQ ID NO: 14) operably linked to a downstream (3′) homology arm (down) to the serA locus (serA.down; SEQ ID NO: 23).

The DNA fragments were amplified using Q5 DNA polymerase per the manufacturer's instructions. The PCR products were purified using Zymo clean and concentrate 5 columns per manufacturer's instructions. The DNA fragments A, B and C were assembled into a plasmid pRS426 purchased from ATCC (ATCC® 77107™) by using yeast gap-repair cloning method (Joska et al., 2014), generating pWS745 (SEQ ID NO: 37)

### Example 7

### Construction of Ws2835 Strain Expressing Amylase 2 with modBli03445ss

In the instant example, the expression cassettes constructed and described in Examples 6 and 7 were integrated into an exemplary B. licheniformis host strain. More particularly, an exemplary B. licheniformis host (WS2835) was constructed by integrating the 1^{st} and 2^{nd} copy Amylase 2 cassettes into a B. licheniformis strain named BF719 (ΔserA ΔlysA). For example, the recombinant B. licheniformis strain (WS2835) was constructed by integration of the 1^{st} Amylase 2 cassette (Amylase 2; SEQ ID NO: 36) into the lysA locus and then the 2^{nd} Amylase 2 cassette (Amylase 2; SEQ ID NO: 38) into the serA locus in BF719 (ΔserA ΔlysA) as described below.

The 1^{st} Amylase 2 cassette (lysA::[p3-modBli03445ss-amylase 2] lysA); SEQ ID NO: 36) integration fragment was generated by PCR amplification from a plasmid template pWS743 (SEQ ID NO: 34) with the ws683 (SEQ ID NO: 16) and ws688 (SEQ ID NO: 17) primer pair. The 2^{nd} Amylase 2 cassette (serA::[p]-modBli03445ss-amylase 2] serA); SEQ ID NO: 38) integration fragment was generated by PCR amplification from a plasmid template pWS745 (SEQ ID NO: 37) with the ws709 (SEQ ID NO: 24) and ws714 (SEQ ID NO: 25) primer pair.

The Amylase 2 expression cassettes were transformed into BF613 strain using the method as described PCT Publication No. WO2019/040412. Briefly, the BF613 competent cells were generated by growing the strain overnight in L broth containing one hundred (100) ppm spectinomycin at 37° C. with 250 RPM shaking. The culture was diluted the next day to an OD₆₀₀ of 0.7 of fresh L broth containing one hundred (100) ppm spectinomycin. This new culture was grown for one (1) hour at 37° C., 250 RPM shaking. D-xylose was added to 0.1% w·v⁻¹. The culture was grown for an additional four (4) hours at 37° C. and 250 RPM shaking. The cells were harvested at 1700-g for seven (7) minutes, and used as competent cells for transformation.

One hundred (100) μl of BF613 competent cells were mixed with twenty (20) μl of the 1^{st}_cassette (SEQ ID NO: 36) integration fragment. The cell/DNA mixture was incubated at 1200 RPM, 37° C. for one and a half (1.5) hours. The mixture was then plated on TSS agar plates containing eighty-eight (88) ppm serine and hundred (100) ppm spectinomycin. The inoculated plates were incubated at 37° C. for forty-eight (48) hours. Transformed colonies were screened by PCR-amplification with the ws775 (SEQ ID NO: 26) and ws776 (SEQ ID NO: 27) primer pair. This PCR product, a 1905 bp fragment (SEQ ID NO: 40), was sequenced using the method of Sanger and the ws775 and ws776 primer pair listed in TABLE 5. A colony with the correct integration of the 1^{st} Amylase 2 cassette (SEQ ID NO: 36) was stored and named WS2834.

The WS2834 competent cells were generated as described above. One hundred (100) μl of WS2834 competent cells were mixed with twenty (20) μl of the 2^{nd} Amylase 2 cassette (SEQ ID NO: 38) integration fragment. The cell/DNA mixture was incubated at 1200 RPM, 37° C. for one and a half (1.5) hours. The mixture was then plated on TSS agar plates. The inoculated plates were incubated at 37° C. for forty-eight (48) hours. Transformed colonies were screened by PCR-amplification with 1617 (SEQ ID NO: 29) and ws717 (SEQ ID NO: 30) primer pair. This PCR product, a 1849 bp fragment (SEQ ID NO: 41), was sequenced using the method of Sanger and the 1617 and ws717 primers. A colony with the correct integration of the 1^{st} Amylase 2 cassette (SEQ ID NO: 36) and 2^{nd} Amylase 2 cassette (SEQ ID NO: 38) was passaged on L agar until the colonies were stored as strain WS2835.

To test a relative performance of the modified B. licheniformis Bli03445 signal sequence (modBli03445ss; SEQ ID NO: 6) on amylase 2 production, a control strain with a modified B. licheniformis AmyL signal sequence (modAmyLss; SEQ ID NO: 4) was constructed in BF613 (ΔserA ΔlysA) by the integration of a 1^{st} Amylase 2 cassette (lysA::p3-modAmyLss-amylase 2 lysA) at the lysA locus and a 2^{nd} Amylase 2 cassette (serA::[p1-modAmyLss-amylase 2] serA) at the serA locus. The 1^{st} Amylase 2 cassette (SEQ ID NO: 42) comprises an upstream homology arm (up) for the lysA locus (lysA.up; SEQ ID NO: 10) operably linked to the DNA sequence encoding the lysA ORF (SEQ ID NO: 11) operably linked to the synthetic p3 promoter (pro; SEQ ID NO: 22) operably linked to DNA encoding a B. subtilis aprE 5′-UTR (SEQ ID NO: 13) operably linked to DNA encoding a modified B. licheniformis AmyL signal sequence (modAmyLss; SEQ ID NO: 4) operably linked to DNA encoding amylase 2 operably linked to the B. licheniformis amyL transcriptional terminator (SEQ ID NO: 14) operably linked to the downstream homology arm (down) for the lysA locus (lysA.down; (SEQ ID NO: 15). The 2^{nd} Amylase 2 cassette (SEQ ID NO: 43) comprises an upstream homology arm (up) to the serA locus (serA.up; SEQ ID NO: 20) operably linked to the serA ORF (SEQ ID NO: 48) operably linked to the synthetic p1 promoter (pro; SEQ ID NO: 39) operably linked to DNA encoding the B. subtilis aprE 5′-UTR (SEQ ID NO: 13) operably linked to the DNA encoding the modified B. licheniformis AmyL signal sequence (modAmyLss; SEQ ID NO: 4) operably linked to DNA encoding amylase 2 operably linked to the B. licheniformis amyL transcriptional terminator (SEQ ID NO: 14) operably linked to the downstream homology arm (down) to the serA locus (serA.down; SEQ ID NO: 23). More particularly, the modified AmyL signal sequence (modAmyLss; SEQ ID NO: 4), relative to the native AmyL signal sequence (AmyLss; SEQ ID NO: 3) as shown in FIG. 2, comprises substitutions of alanine (A) to serine (S) at the ⁻2 position (i.e., relative to the signal peptidase cleavage site).

### Example 8

### Effect of the Modified B. Licheniformis Bli03445 Signal Sequence (Bli03445ss) on Amylase 2 Production

In the present example, the WS2835 strain comprising two (2) copies of Amylase 2 expression cassettes with the modified B. licheniformis Bli03445 signal sequence (modBli03445ss; SEQ ID NO: 6) were assayed for production of the amylase reporter protein compared to the control LDN573-8 strain comprising two (2) copies of Amylase 2 expression cassettes with the modified B. licheniformis AmyL signal sequence (modAmyLss; SEQ ID NO: 4), using standard small-scale conditions, as described in PCT Publication No. WO2018/156705 and WO2019/055261 (each incorporated herein by reference). Alpha-amylase production was quantified using the method of Bradford or the Ceralpha assay, wherein the relative improvement in production of Amylase 2 of the WS2835 strain is compared to the control strain (LDN573-8) is presented below TABLE 9.

TABLE 9
Relative Performance of modBli03445ss
vs. modAmyLss on Amylase 2 Production

| | | | Relative Amy2 |
|---|---|---|---|
| Strain Name | Signal Sequence (ss) | SEQ ID NO | Production |
| WS2835 | modBli03345ss | 6 | 1.19 |
| LDN573-8 | modAmyLss | 4 | 1.00 |

As presented in TABLE 9, the modified B. licheniformis Bli03445 signal sequence (modBli03445ss) demonstrates a significant improvement in Amylase 2 reporter protein production in the WS2835 strain, relative to the B. licheniformis (control) strain LDN573-8 comprising the modified AmyL signal sequence (modAmyLss).

### REFERENCES

PCT Publication No. WO2002/14490
PCT Publication No. WO2003/08312
Ausubel et al., “Current Protocols in Molecular Biology”, published by Greene Publishing Assoc. and Wiley-Interscience (1987).
Brode et al., “Subtilisin BPN′ variants: increased hydrolytic activity on surface-bound substrates via decreased surface activity”, Biochemistry, 35(10):3162-3169, 1996.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Earl et al., “Ecology and genomics of Bacillus subtilis”, Trends in Microbiology, 16(6):269-275, 2008.
Ferrari et al., “Genetics,” in Harwood et al. (ed.), Bacillus, Plenum Publishing Corp., 1989.
Joska et al., J of Microbiol. Methods, 2014, 100: 46-51.
Olempska-Beer et al., “Food-processing enzymes from recombinant microorganisms—a review” Regul. Toxicol. Pharmacol., 45(2):144-158, 2006.
Raul et al., “Production and partial purification of alpha amylase from Bacillus subtilis (MTCC 121) using solid state fermentation”, Biochemistry Research International, 2014.
Sambrook et al., “Molecular Cloning: A Laboratory Manual” Cold Spring Harbor Laboratory: Cold Spring Harbor, N.Y. (1989), (2001) and (2012).
Van Dijl and Hecker, “Bacillus subtilis: from soil bacterium to super-secreting cell factory”, Microbial Cell Factories, 12(3). 2013.
Westers et al., “Bacillus subtilis as cell factory for pharmaceutical proteins: a biotechnological approach to optimize the host organism”, Biochimica et Biophysica Acta., 1694:299-310, 2004.

1. A nucleic acid encoding a signal sequence comprising SEQ ID NO: 2.

2. A nucleic acid encoding a signal sequence comprising SEQ ID NO: 6.

3. A polynucleotide comprising an upstream (5′) nucleic acid encoding a signal sequence comprising SEQ ID NO: 2 operably linked to a downstream (3′) nucleic acid encoding a protein of interest (POI).

4. A polynucleotide comprising an upstream (5′) nucleic acid encoding a signal sequence comprising SEQ ID NO: 6 operably linked to a downstream (3′) nucleic acid encoding a protein of interest (POI).

5. A polynucleotide comprising an upstream (5′) promoter operably linked to a downstream nucleic acid encoding a signal sequence comprising SEQ ID NO: 2 operably linked to a downstream (3′) nucleic acid encoding a protein of interest (POI).

6. A polynucleotide comprising an upstream (5′) promoter sequence operably linked to a downstream nucleic acid encoding a signal sequence comprising SEQ ID NO: 6 operably linked to a downstream (3′) nucleic acid encoding a protein of interest (POI).

7. (canceled)

8. The polynucleotide of claim 3, wherein the nucleic acid encoding the POI encodes an enzyme.

9. (canceled)

10. A recombinant Bacillus cell comprising at least two introduced polynucleotides of claim 5.

11-12. (canceled)

13. A recombinant Bacillus cell expressing at least two introduced polynucleotides encoding a heterologous protein of interest (POI), wherein the introduced polynucleotides comprises an upstream (5′) nucleic acid encoding a signal sequence comprising SEQ ID NO: 2 operably linked to a downstream (3′) nucleic acid encoding the POI.

14-15. (canceled)

16. A recombinant Bacillus cell expressing at least two introduced polynucleotides encoding a heterologous protein of interest (POI), wherein the first and second introduced polynucleotides comprise an upstream (5′) nucleic acid encoding a signal sequence comprising SEQ ID NO: 2 operably linked to a downstream (3′) nucleic acid encoding the POI, and an upstream (5′) nucleic acid encoding a signal sequence comprising SEQ ID NO: 6 operably linked to a downstream (3′) nucleic acid encoding the POI. respectively.

17. A method for expressing a heterologous protein of interest (POI) in a Bacillus cell comprising: (a) obtaining or constructing a Bacillus cell comprising an introduced polynucleotide comprising an upstream (5′) promoter operably linked to a downstream nucleic acid encoding a modified SacB signal sequence (modSacBss) comprising SEQ ID NO: 2 operably linked to a downstream (3′) nucleic acid encoding the POI, and (b) fermenting the Bacillus cell under suitable conditions for the expression of the POI.

18-20. (canceled)

21. A method for expressing a heterologous protein of interest (POI) in a Bacillus cell comprising: (a) obtaining or constructing a Bacillus cell comprising an introduced polynucleotide comprising an upstream (5′) promoter operably linked to a downstream nucleic acid encoding a modified Bli03445 signal sequence (modBli03445) comprising SEQ ID NO: 6 operably linked to a downstream (3′) nucleic acid encoding the POI, and (b) fermenting the Bacillus cell under suitable conditions for the expression of the POI.

22-24. (canceled)
